(19) United States
(12) Patent Application Publication
(10) Pub. No.: US 2022/0133834 A1
(43) Pub. Date: May 5, 2022

(54) Composition For Inhibiting Proliferation of Influenza Virus

(71) Applicant: Max Probio Co., Ltd., Shiga (JP)

(72) Inventors: Kazuo Maehara, Shiga (JP); Kyoko Hayashi, Toyama (JP); Yoshiteru Maehara, Shiga (JP); Toshihiro Kurusu, Nagano (JP); Taizo Watanabe, Kyoto (JP); Kenji Asaoka, Shiga (JP)

(21) Appl. No.: 17/519,252

(22) Filed: Nov. 4, 2021

(30) Foreign Application Priority Data
Nov. 4, 2020 (JP) 2020-184225

(51) Int. Cl. A61K 36/899; A61P 31/16

(52) U.S. Cl. CPC A61K 36/899; A61P 31/16

(57) ABSTRACT

A composition for inhibiting proliferation of influenza virus has fermented rice bran powder produced using non-pathogenic microbes that include, as cellulolytic bacteria, at least three Bacillus species, Bacillus amyloliquefaciens M-4 (deposit number: NITE P-02538 and NITE BP-02538), Bacillus subtilis M-5 (deposit number: NITE P-02539 and NITE BP-02539) and the Bacillus subtilis-related species Bacillus sp. M-6 (deposit number: NITE P-02940 and NITE BP-02940), and at least one Paenibacillus species, Paenibacillus cineris M-9 (deposit numbers: NITE AP-03304 and NITE BP-03304).

### FIELD OF THE INVENTION

The present invention relates to a composition for inhibiting proliferation of influenza virus.

### BACKGROUND OF THE INVENTION

In the past, the present inventors have proposed the invention of Japanese Patent No. 6596135 as a composition for improving the enteral environment and reducing weight gain caused by ingestion of a high fat diet, or for inhibiting Helicobacter pylori bacteria.

### SUMMARY OF THE INVENTION

In recent years, greater importance has been placed on the strategy of artificially augmenting the natural biological function of protecting against infection (immunity) as a measure against influenza. Influenza viruses are categorized as type A, type B or type C. However, only types A and B infect humans to cause influenza. Both viruses are RNA viruses having genomes of negative single-stranded RNA segmented into 8 strands. Among these influenza viruses, the influenza A virus has a structure known as a nucleocapsid, composed of a gene and a protein shell encapsulating it, with an envelope on its outer side, and having the characteristic glycoproteins hemagglutinin (HA) and neuraminidase (NA) protruding as spikes on the envelope. These have 16 and 9 subtypes, respectively, and gene recombination in infected cells leads to the appearance of influenza A viruses with major mutations. One example is the new swine flu virus (H1N1 subtype) that was responsible for the epidemic of 2009, where the virus gene was a combination of swine, bird and human influenza viruses.

During the course of proliferation of influenza virus, the virus binds to (adsorbs onto) the surfaces of host cells, and whole viral particles are taken up into cells by endocytosis (the stage known as invasion). The viral particle membranes are then ruptured allowing the gene (RNA) to leave (decapsulation). Proteins (enzymes) necessary for proliferation of viral descendants (replication) are synthesized based on the genetic information, and biomolecules such as sugars and lipids in the cell are also used to form new viral particles. The newly formed viral particles migrate into the cell and protrude onto the host cell surface, and the enzyme neuraminidase (NA) of the viruses that have been synthesized in the cells cause the viral particles to be cleaved from the cell membrane (release). The descendant viruses that have been released out of the cell adsorb and infiltrate into nearby uninfected cells, undergoing further repeated proliferation and resulting in production of large amounts of the virus. Infection and proliferation of a virus activates immunomechanisms that recognize the virus as foreign to the body and work to eliminate it, causing inflammatory and fever responses. Typical symptoms of influenza include fever of 38° C. or higher, nasal obstruction, arthralgia, muscular pain, general malaise and loss of appetite.

Anti-influenza drugs that are approved in Japan and commonly used in the clinic include neuraminidase inhibitors such as oseltamivir (Tamiflu), zanamivir (Relenza), laninamivir (Inavir) and peramivir (Rapiacta), and the cap-dependent endonuclease selective inhibitor baloxavir (Xofluza). However, the use of such chemically synthesized drugs targeting enzymes involved in viral proliferation is associated with the serious problem of causing mutation of the virus toward a form in which the effects of the drugs are lowered (drug-resistant virus), and such inhibitors have therefore had limited effectiveness.

Inoculation with vaccines has also been developed as a countermeasure against influenza, but since the virus gene mutates frequently it is necessary to engage in vaccine production for each mutated form. The purpose of vaccination is to cause production of anti-influenza virus antibodies in the body, but when immunity is compromised, such as in the case of elderly or patients with underlying disease, antibody production ability is weakened and it may not be possible to guarantee a sufficient amount of antibody.

Greater importance has therefore been placed on the strategy of artificially augmenting the natural biological function of protection against infection (immunity) as a measure against influenza.

The present inventors have diligently examined the technical problem of inhibiting influenza virus, and have found as a result that it is possible to inhibit influenza virus by modifying the invention described in Patent Document 1.

Specifically, it is an object of the present invention to provide a composition for inhibiting proliferation of influenza virus, which is able to inhibit influenza virus.

In order to achieve the object stated above, the composition for inhibiting proliferation of influenza virus according to the present invention comprises fermented rice bran powder produced using non-pathogenic microbes that include, as cellulolytic bacteria, at least three Bacillus species, Bacillus amyloliquefaciens M-4 (deposit number: NITE P-02538 and NITE BP-02538), Bacillus subtilis M-5 (deposit number: NITE P-02539 and NITE BP-02539) and the Bacillus subtilis-related species Bacillus sp. M-6 (deposit number: NITE P-02940 and NITE P-02940), and at least one Paenibacillus species, Paenibacillus cineris M-9 (deposit numbers: NITE AP-03304 and NITE BP-03304).

According to the present invention, it is possible to inhibit the proliferation of influenza virus.

### BRIEF DESCRIPTION OF THE DRAWINGS

FIG. 1 is a schematic diagram showing a simplified molecular phylogenetic tree based on a partial nucleotide sequence of 16S rDNA, obtained by analysis of Bacillus amyloliquefaciens M-4 included in the non-pathogenic microbes in the composition for inhibiting influenza virus according to the present invention.

FIG. 2 is a schematic diagram showing a similar simplified molecular phylogenetic tree obtained by analysis of Bacillus subtilis M-5 included in the same non-pathogenic microbes.

FIG. 3 is a schematic diagram showing a similar simplified molecular phylogenetic tree obtained by analysis of Bacillus sp. M-6 included in the same non-pathogenic microbes.

FIG. 4 is a schematic diagram showing a similar simplified molecular phylogenetic tree obtained by analysis of Paenibacillus cineris M-9 included in the same non-pathogenic microbes.

FIG. 5 is a correlation diagram for body weight-days elapsed, showing transition of body weight increase in mouse groups with and without administration of the same composition for inhibiting influenza virus.

FIG. 6 is a graph showing the level of virus in the lungs of the same mouse groups 3 days after infection.

FIG. 7 is a graph showing neutralizing antibody levels in bronchoalveolar lavage fluid of the same mouse group 14 days after infection.

FIG. 8 is a graph showing neutralizing antibody levels in serum of the same mouse groups 14 days after infection.

FIG. 9 is a graph showing IgA levels in feces of the same mouse groups.

FIG. 10 is a graph showing IgA levels in bronchoalveolar lavage fluid of the same mouse groups.

FIG. 11 is a graph showing IFN-γ levels in serum of the same mouse groups.

FIG. 12 is a graph showing IFN-γ levels in lungs of the same mouse groups.

FIG. 13 is a graph showing IFN-γ levels in spleens of the same mouse groups.

### DETAILED DESCRIPTION OF THE PREFERRED EMBODIMENTS

The composition for inhibiting proliferation of influenza virus according to the present invention, which is based on thorough consideration given to the biological features of different microorganisms, is prepared with careful selection of the types and combinations of microorganisms added to rice bran powder so that heterogenous microbes can exhibit their maximum bioactivity in a fermented rice bran product with satisfactory interaction between the microbes, thereby eliminating problems that can harm human or animal health. In other words, the composition for inhibiting proliferation of influenza virus comprises a fermented product obtained by fermentation of rice bran powder using non-pathogenic microbes that include, as cellulolytic bacteria, at least four novel species: Bacillus amyloliquefaciens M-4, Bacillus subtilis M-5, the Bacillus subtilis-related species Bacillus sp. M-6 and the Paenibacillus species, Paenibacillus cineris M-9.

The four cellulolytic bacteria, Bacillus amyloliquefaciens M-4 (hereunder abbreviated to “species M-4”), Bacillus subtilis M-5 (hereunder abbreviated to “species M-5”), Bacillus sp. M-6 (hereunder abbreviated to species M-6) and Paenibacillus cineris M-9 (hereunder abbreviated to “species M-9”) are all novel bacteria discovered by the present inventors, and have been deposited at the National Institute of Technology and Evaluation (NITE)/NITE Patent Microorganisms Depositary (NPMD), having an address of 2-5-8, Kazusakamatari, Kisarazu-shi, Chiba 292-0818 Japan. Species M-4 was originally deposited on Aug. 29, 2017 and was assigned deposit accession number NITE P-02538 (NITE APB-02538) and upon a request to convert such original deposit to a deposit under the Budapest Treaty filed Sep. 27, 2021, such deposit of species M-4 was assigned accession number NITE BP-02538 by NITE/NPMD, on Nov. 8, 2021. Species M-5 was originally deposited on Aug. 29, 2017 and was assigned deposit accession number NITE P-02539 (NITE APB-02539) and upon a request to convert such original deposit to a deposit under the Budapest Treaty filed Sep. 27, 2021, such deposit of species M-5 was assigned accession number NITE BP-02539 by NITE/NPMD, on Nov. 8, 2021. Species M-6 was originally deposited on Aug. 29, 2017 and was assigned deposit number NITE P-02540 (NITE APB-02540) and upon a request to convert such original deposit to a deposit under the Budapest Treaty filed Sep. 27, 2021, such deposit of species M-6 was assigned accession number NITE BP-02540 by NITE/NPMD, on Nov. 8, 2021. Species M-9 was originally deposited on Oct. 23, 2020 and was assigned deposit number NITE AP-03304, and upon a request to convert such original deposit to a deposit under the Budapest Treaty filed Sep. 27, 2021, such deposit of species M-9 was assigned accession number NITE BP-03304 by NITE/NPMD, on Oct. 12, 2021.

DNA identification analysis has confirmed the novel Bacillus species M-4, species M-5 and species M-6 as Bacillus and species M-9 as Paenibacillus. The method of identification used was the following.

DNA extraction: Achromopeptidase (Wako Pure Chemical Industries, Japan)

PCR amplification: PrimeSTAR HS DNA Polymerase (Takara Bio, Japan)

Cycle sequencing: BigDye Terminator v3.1 Cycle Sequencing Kit (Applied Biosystems, USA)

Nucleotide sequencing: ChromasPro 1.7 (Technelysium, AUS)

Software: TechnoSuruga Lab Microbial Idenfitication System (TechnoSuruga Laboratory, Japan)

Database: DB-BA11.0 (TechnoSuruga Laboratory)

International nucleotide sequence database (DDBJ/ENA (EMBL)/GenBank)

Identification: Analysis based on 16S rDNA partial nucleotide sequence

The following results were obtained by this identification method using analysis of the 16S rDNA partial nucleotide sequence of the aforementioned three Bacillus bacterial species and the one Paenibacillus bacterial species.

### [Species M-4]

Identified species: Bacillus amyloliquefaciens, Biosafety level 1.
Highest homology by BLAST search (reference strain): Bacillus amyloliquefaciens subsp. amyloliquefaciens NBRC 15535^{T} . . . 99.8% homology.

### [Species M-5]

Identified species: Bacillus subtilis-related Bacillus sp., Biosafety level 1
Highest homology by BLAST search (reference strain): Bacillus subtilis subsp. subtilis DSM 10^{T} . . . 99.6% homology.

### [Species M-6]

Identified species: Bacillus subtilis-related Bacillus sp., Biosafety level 1
Highest homology by BLAST search (reference strain): Bacillus subtilis subsp. subtilis DSM 10^{T} . . . 99.6% homology.

### [Species M-9]

Identified species: Paenibacillus cineris, Biosafety level 1.
Highest homology by BLAST search (reference strains): Paenibacillus favisporus GMP01^{T}, Paenibacillus cineris LMG 18439^{T} and Paenibacillus celluositrophicfus P2-1^{T} . . . 99.6% homology.

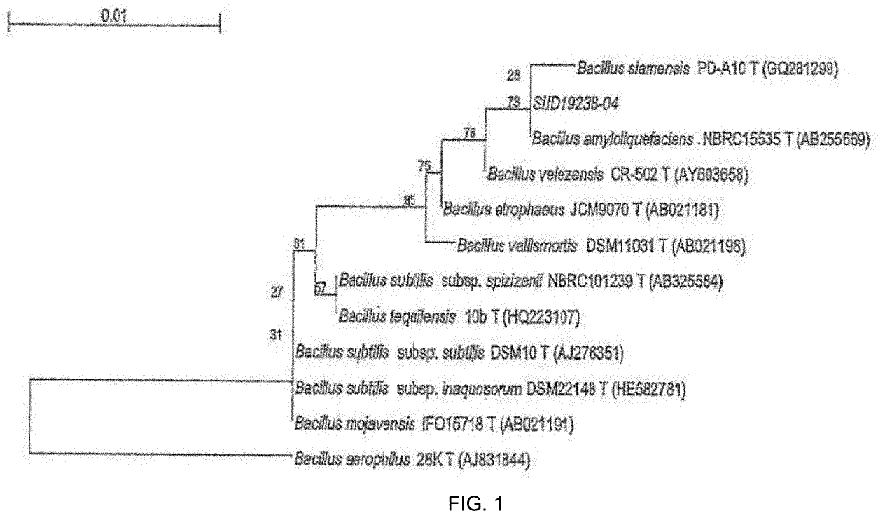

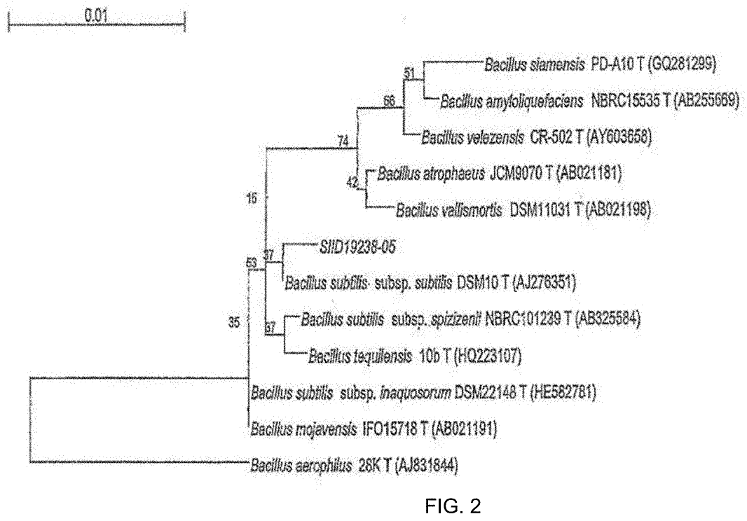

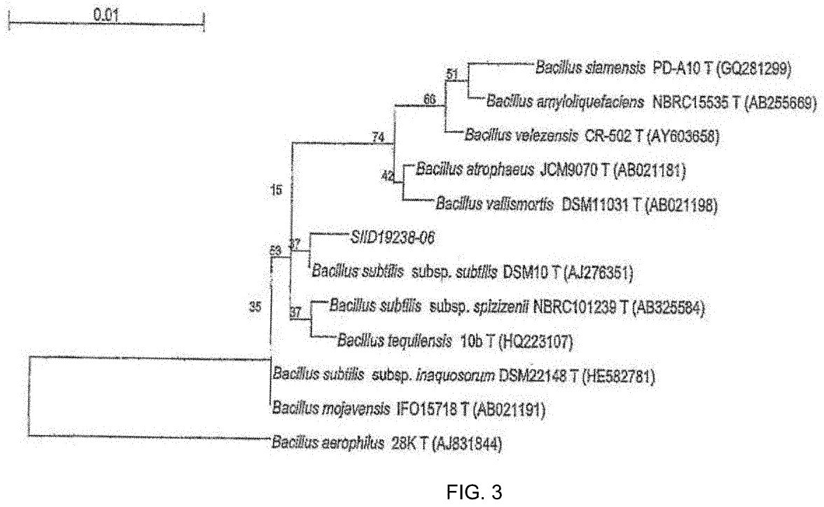

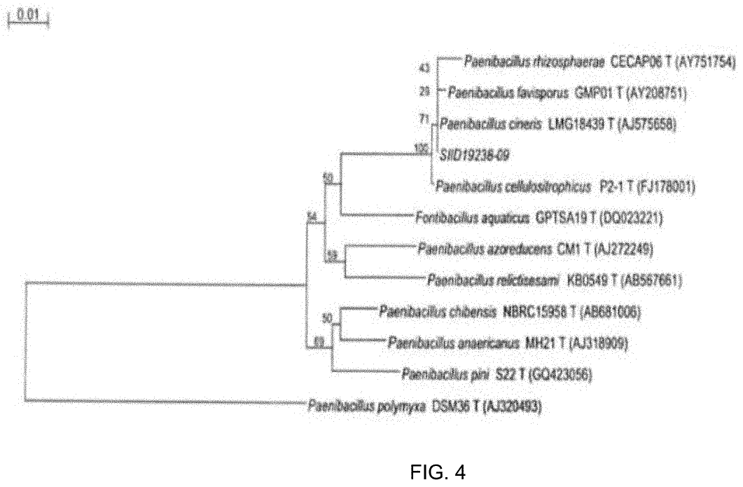

Simplified molecular phylogenetic trees of the three Bacillus and one Paenibacillus bacteria based on their respective 16S rDNA partial nucleotide sequences are shown in FIG. 1 to FIG. 4. SIID19238-04 in FIG. 1 represents species M-4, SIID19238-05 in FIG. 2 represents species M-5, SIID19238-06 in FIG. 3 represents species M-6 and SIID19238-09 in FIG. 4 represents species M-9. FIG. 1 to FIG. 4 show scale bars at the top left, bootstrap values as numerals located at the phylogenetic branches, and Type strains as T at the ends of the strain names, respectively.

For production of a composition for inhibiting proliferation of influenza virus according to the present invention, the necessary live bacterial culture solutions containing at least the aforementioned 4 microbes stored in their respective pure cultures are prepared and added to rice bran powder either separately or as a mixture at ordinary temperature in the form of a spray, for example, and the rice bran powder is thoroughly stirred and mixed by manual or mechanical means to fully disperse all of the live bacteria throughout the rice bran, after which a sugar solution such as glucose solution is added in a suitable amount as a bacterial nutrient and the mixture is stirred to uniformity, and stored for a prescribed time (usually about 1 to 4 months) in a fermenter for fermentation. In order to prevent infiltration of external bacteria during storage, the open section of the fermenter is covered with a cover such as a vinyl sheet, while naturally also carrying out shielding measures for the room where the fermenter is installed, in order to prevent inadvertent inclusion of contaminating external air. Since the temperature of the rice bran gradually increases as fermentation proceeds, the temperature is continuously measured and controlled to a suitable temperature (such as ≤50° C.), to prevent alteration of the effective components, or death of useful bacteria that are susceptible to high temperatures. Temperature decrease is usually carried out in an air-cooling system, by removing the cover and exposing the content to indoor air for a fixed period.

The rice bran powder that has reached a sufficient fermented state is thus lowered in temperature, after which it is allowed to naturally dry for several weeks after passing through a pulverizer, and packed into a predetermined container for use as a product. The fermented rice bran product can be ingested directly through the mouth, or since it is in powder form it may be added to beverages such as juices or sprinkled onto food or mixed into cooking materials to further facilitate ingestion. It can be suitably used not only as a human health food but also as a compounding agent for feeds used to support racehorse, livestock or pet health.

Oral ingestion of a composition for inhibiting proliferation of influenza virus can thus inhibit influenza virus.

### Examples

The present inventors conducted the following experiments to determine whether or not the composition for inhibiting proliferation of influenza virus of the present invention is able to inhibit influenza virus.

### [Experiment Conditions]

<Virus Strain>

Influenza A virus (A/NWS/33, H1N1 subtype)

<Experimental Animals>

BALB/c mice (female, 6 weeks old)

Of 10 mice in each group, five (group A) were examined by sampling the feces, whole blood, lungs, bronchoalveolar lavage fluid (BALF) and spleen 3 days after infection. The remaining five (group B) had their daily body weights and deaths recorded during the period from the day of infection until 14 days afterward, and the whole blood, lungs, bronchoalveolar lavage fluid, spleen and feces were sampled after 14 days.

<Administered Group>

1. First sample (distilled water, administration period: day of infection until 14 days afterward)

2. Second sample (Tamiflu, dose: 0.2 mg/0.4 mL/day, administration period: day of infection until 14 days afterward)

3. Third sample (MAX composition (group administered one month prior), dose: 20 mg/0.4 mL/day, administration period: day of infection until 14 days afterward)

4. Fourth sample (MAX composition (group administered 2 months prior), dose: 20 mg/0.4 mL/day, administration period: day of infection until 14 days afterward)

<Feed>

The third sample and fourth sample were switched with NMF feed (high fiber feed, fiber content: 4.4%, product of Oriental Yeast Co., Ltd.) during the period from the day of sample administration until the final day of the experiment (14 days after infection). For the first sample and second sample, ordinary feed (CRF-1, product of Oriental Yeast Co., Ltd.) was provided.

The MAX composition was produced in the following manner. Specifically, the inoculation used was a mixture of MAX source bacteria containing the following pure cultured and prolonged-storage bacterial strains, both from Matsumoto Institute of Microorganism Co., Ltd. The inoculum contained four strains of cellulolytic bacteria, which were the aforementioned novel Bacillus and Paenibacillus bacteria.
Cellulolytic bacteria: Bacillus amyloliquefaciens M-4 (deposit number: NITE P-02538), Bacillus subtilis M-5 (deposit number: NITE P-02539), Bacillus sp. M-6 (deposit number: NITE P-02540), Paenibacillus cineris M-9 (deposit numbers: NITE AP-03304 and NITE BP-03304)
Lactic acid bacteria: Lactobacillus casei NBRC15883
Bifidobacteria: Bifidobacterium bifidum NBRC100015
Aspergillus: Aspergillus oryzae NBRC6215

To 300 kg of rice bran powder with a mean particle size of ≤1 mm there were added 30 Kg of the aforementioned MAX source bacteria mixture (cell count: ˜10¹⁰ per 1 g, with species M-4, species M-5, species M-6 and species M-9 constituting approximately 70% of the total cell count), with repeated manual mixing, to thoroughly disperse the bacteria throughout the entire rice bran powder. Next, 115 L of a 0.06% glucose solution was slowly added to the bacteria-containing rice bran powder in portions while uniformly mixing by manual stirring, to prepare a fermentation starting material. The fermentation starting material was filled into a 1000 L fermenter with an open top, and after about 1 month of storage while continuously controlling the temperature to maintain a starting material temperature of ≤50° C. with the top covered with a vinyl sheet, for sufficient fermentation, the obtained fermented rice bran powder was passed through a pulverizer and allowed to naturally dry for 2 weeks to produce a MAX composition.

### [Experiment Method]

(1) On the starting day of oral administration of the sample, stools were collected from the mice in group B and provided for IgA analysis. Oral administration of the sample was then initiated.
(2) During the period from 2 months before viral infection (fourth sample), 1 month before viral infection (third sample) or on the day of infection (first sample and second sample) until 14 days afterward, the sample was orally administered twice per day (9 am and 6 pm) to the 10 mice in each group of BALB/c mice. For the third sample and fourth sample, the feed was switched to NMF when oral administration was initiated.
(3) On the day of infection (0 d), the virus (2×10⁴ PFU/50 μl/mouse) was nasally administered to each mouse under anesthesia. PFU stands for Plaque Forming Units.
(4) The body weights and deaths were recorded (for group B alone) on the 14th day after infection.
(5) On the third day after infection, samples of bronchial lavage fluid (for measurement of virus and IgA levels), lungs (for measurement of virus and IFN-γ levels), whole blood (separating the serum) and spleen (for measurement of IFN-γ levels) were taken from 5 mice of each group (group A). IFN-γ stands for interferon-γ.
(6) At 14 days after infection, feces (for measurement of IgA levels), whole blood (separating the serum), spleen, bronchial lavage fluid and lungs were sampled from the remaining (5) mice of each group (group B).
(7) Sampling period

First sample and second sample: Twice on first day of oral administration, day of viral infection and 14 days after infection

Third sample: Three times at start of oral administration, day of viral infection and 14 days after infection

Fourth sample: Five times on first day of oral administration (day 0), 15 days after administration, 30 days after administration, 60 days after administration (day of viral infection) and 74 days after administration (14 days after viral infection)

### [Measured Parameters and Measuring Methods]

(1) Recording of body weights and deaths (day of infection to 14 days after infection)
(2) Virus levels (Plaque assay method: lungs and bronchoalveolar lavage fluid 3 days after infection)

<Measurement of Virus Level by Plaque Assay Method>

The sample was diluted 10⁰, 10¹, 10², 10³, 10⁴ and 10⁵-fold with PBS (phosphate buffered saline) and added at 100 μl each to MDCK cells (canine kidney cells) that had been cultured in a single layer on a 35-mm dish, for infection at room temperature for 1 hour. Plaque medium (0.5% ME Agarose-added MEM medium) was then laid over it. After culturing for 2 days in a CO² incubator at 37° C. and confirming appearance of plaques, crystal violet solution was added for fixing and staining of the cells. The number of plaques was measured under a stereomicroscope.

(3) Measurement of neutralizing antibody titer: Serum and bronchoalveolar lavage fluid 14 days after infection

<Measuring Method>

Whole blood was centrifuged (3,000 rpm, 5 minutes, 4° C.) to separate the serum, while the bronchial lavage fluid was centrifuged (1,500 rpm, 5 minutes, 4° C.) and the supernatant was recovered and diluted 1 to 50,000-fold with PBS. To 100 μl of the diluted solution there was mixed 100 μl of the same virus used for infection (2000 PFU/ml). As a control, PBS was added instead of serum or bronchial lavage fluid. After treating the liquid mixture at 37° C. for 1 hour, it was added at 100 μl each to MDCK cells that had been cultured as a single layer on a 35-mm dish, for infection at room temperature for 1 hour. Agar medium was then laid over it and culturing was carried out at 37° C. for 2 days. Upon confirming appearance of plaques, the medium was removed and the cells were fixed and stained with crystal violet solution. The number of plaques was measured under a microscope. The number of plaques of each diluted solution was calculated as a percentage with respect to 100% as the number of plaques in the control. The serum dilution factor that produced 50% inhibition of plaque formation was determined on a graph and the numerical value was recorded as the neutralizing antibody titer.

(4) IgA assay (ELISA): Feces sampled during the period specified above, and bronchoalveolar lavage fluid after 3 days and 14 days

<Measuring Method>

A commercially available assay kit (Mouse IgA ELISA Kit, Bethyl Co.) was used for measurement by the prescribed procedure.

(5) Interferon-γ assay (ELISA): Serum, lungs and spleen 3 days and 14 days after infection

<Measuring Method>

A commercially available assay kit (IFN-GAMMA ELISA Kit, PGI Proteintech Group) was used for measurement by the prescribed procedure.

### [Experimental Results and Considerations]

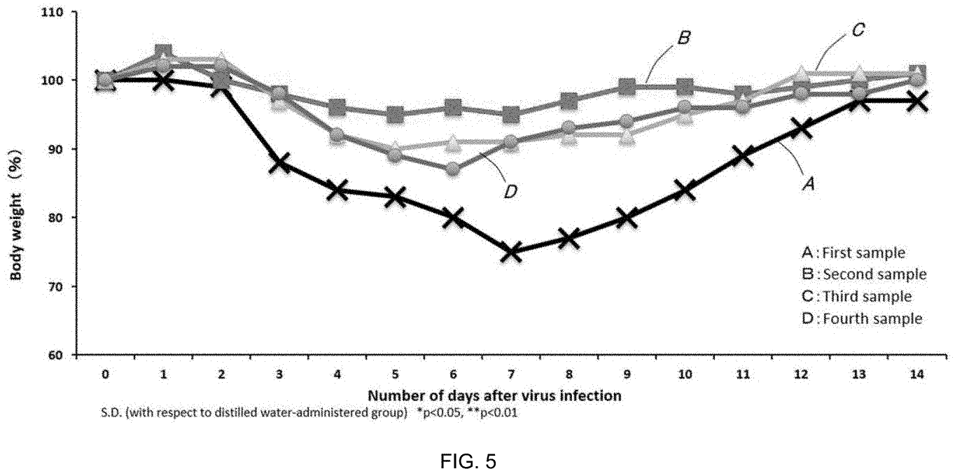

FIG. 5 to FIG. 13 show the results of experimentation conducted with the experiment conditions and methods described above, based on the measured parameters and measuring methods.

### <Changes in Deaths and Body Weights>

There were no mouse deaths in any of the administered groups during the entire administration period. As shown in FIG. 5, for the first sample, the body weights slowly reduced during the first week after infection and gradually recovered after 8 days from infection, returning almost to the original body weights by 2 weeks after infection. For the second sample, almost no reduction in body weight was seen during 2 weeks after infection. With the third and fourth samples, body weight reduction was less overall compared to the first sample, the body weight reduction being inhibited significantly during the period from 4 days to 11 days after infection.

Thus, infection progressed without death of the mice with a viral inoculation dose of 2×10⁴ PFU. The virus level in the lungs was maximum at 3 days after virus inoculation, and body weight decreased thereafter due to loss of appetite accompanying lung inflammation. Inoculation of the mice with influenza virus resulted in body weight reduction as a symptom of infection, without causing symptoms such as sneezing or nasal discharge.

This suggests that the inhibited body weight reduction with MAX composition is an indicator of moderate influenza symptoms.

The average body weight of the mice at the start of the experiment with the first sample (60 days prior to virus inoculation) was 17.2 g, and 20.5 g at virus inoculation, and therefore the body weight increased by 19%. In contrast, with the third sample, the average body weight of the mice at the start of the experiment (60 days prior to virus inoculation) was 17.3 g, and 19.0 g at virus inoculation, and therefore the body weight increased by 10%. With the fourth sample, body weight increased from 17.2 g to 19.4 g, for an increase of 13%. However, since no abnormalities were seen with the mice with the third sample and fourth sample, this inhibition of weight gain may reflect the previously reported diet effect of MAX composition.

<Virus Level in Lungs 3 Days after Infection>

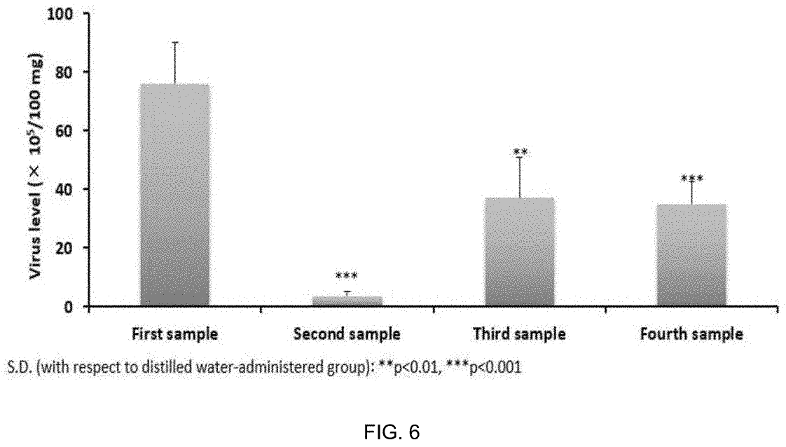

When influenza virus is nasally inoculated into mice in an amount of 50 μL, the virus proliferates in the lungs, the amount reaching a maximum after 3 days. When the virus level in the lungs was measured 3 days after infection, the second sample notably inhibited proliferation, as shown in FIG. 6. With the third sample and fourth sample, on the other hand, significant inhibition of virus proliferation was exhibited in comparison to the first sample.

This leads to the conclusion that virus proliferation in the lungs is inhibited by MAX composition, resulting in alleviated symptoms and consequently inhibited loss of appetite.

<Neutralizing Antibody Titer 14 Days after Infection>

In the bodies of mice infected with influenza virus, antibodies begin to be produced against the virus, with the antibody level rapidly increasing from 2 to 4 weeks after infection. The antibodies produced during this period are specific for all of the constituent proteins of influenza virus, and those antibodies that inhibit infection of the virus by cellular binding are referred to as “neutralizing antibodies.” The most important neutralizing antibodies are antibodies against hemagglutinin (HA) present on the viral envelope. In this experiment, serum and bronchoalveolar lavage fluid were used for measurement of neutralizing antibodies, appropriately diluting them and reacting with virus solutions to determine the amount of virus with which infectivity was still maintained (that is, with which infectivity could not be prevented by antibodies).

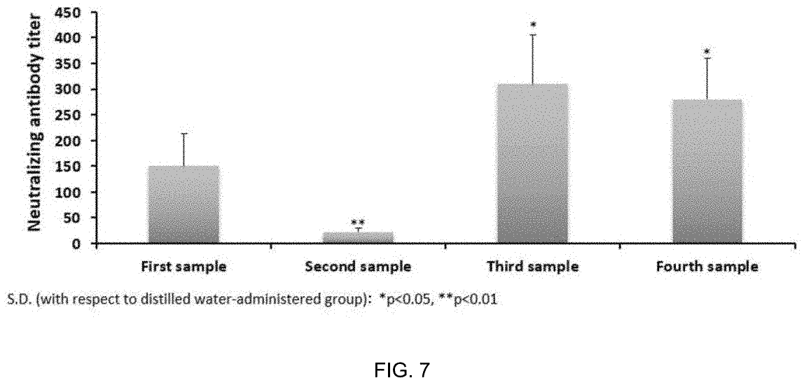

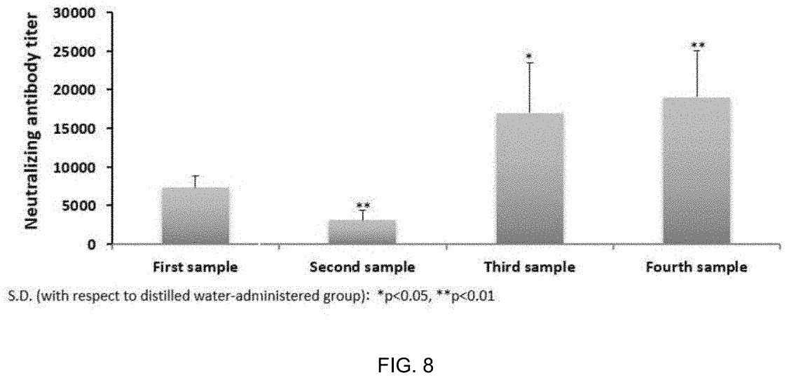

As a result, significantly higher neutralizing antibody was found with the MAX composition compared to the first sample and second sample, both locally (bronchoalveolar lavage fluid) as shown in FIG. 7 and systemically (serum) as shown in FIG. 8. The second sample instead exhibited significantly lower neutralizing antibody, but this is attributed to the exceedingly low viral level in the body which was insufficient to stimulate antibody production, as shown in FIG. 6. In contrast, the MAX composition was shown to have an effect of stimulating antibody production while also suppressing viral load in the body.

### <Total IgA Level in Feces and Bronchoalveolar Lavage Fluid>

Because influenza virus proliferates in the respiratory mucosa, IgA antibodies secreted from mucous membranes are necessary to inhibit its proliferation. Secreted IgA levels were therefore measured in the intestinal tract that is thought to be stimulated by oral administration of MAX composition, and in the bronchi where virus proliferation occurs.

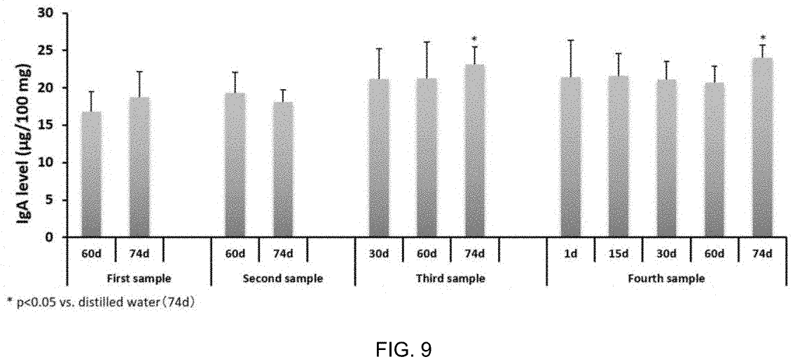

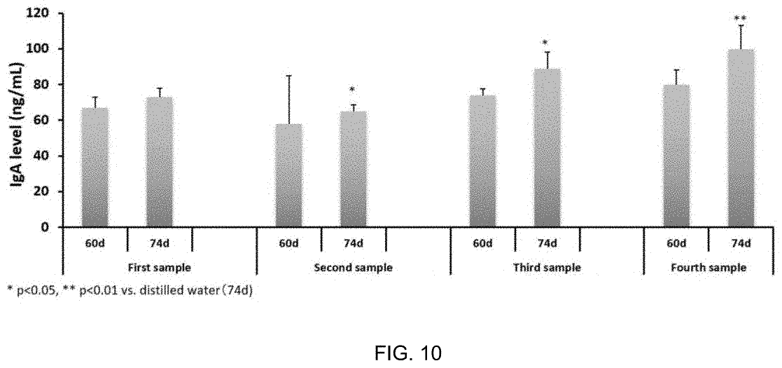

As shown in FIG. 9 and FIG. 10, no significant change in IgA levels was observed with the third sample and fourth sample compared to the first sample and second sample, prior to viral infection. However, as also shown in FIG. 9 and FIG. 10, the IgA levels increased significantly 2 weeks after viral infection (74 days after administration). The increase in IgA with the third and fourth samples at the 74th day is believed to reflect the increase in levels of IgA specific for influenza virus, which is consistent with the increase in neutralizing antibodies (considered to be essentially secreted IgA) in the bronchi shown in FIG. 7.

Moreover, the increase in secreted IgA suggests that these can potentially protect against infection by other viruses such as coronavirus and norovirus for which the antibodies are known to contribute to protection against infection.

### <IFN-γ Levels in Serum, Lungs and Spleen>

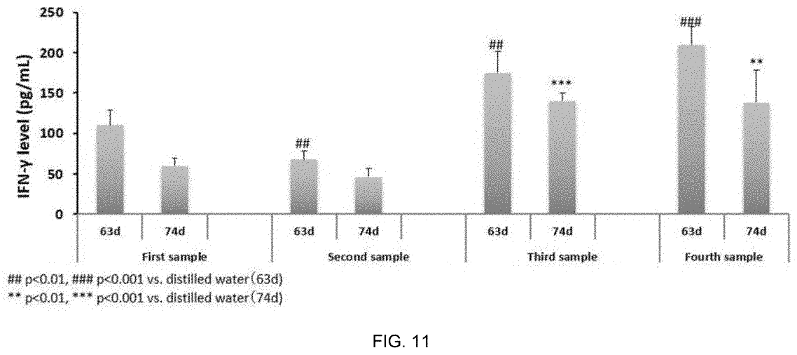

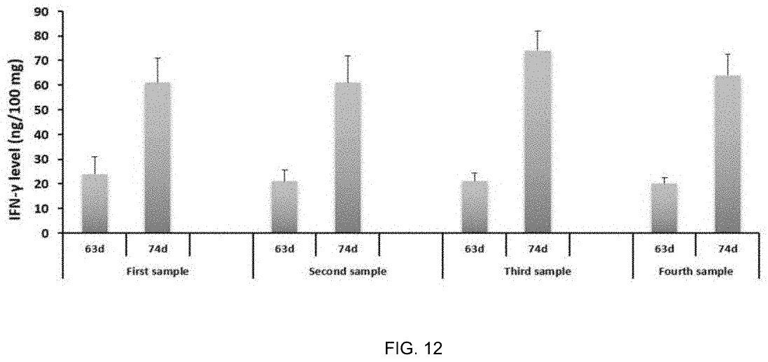

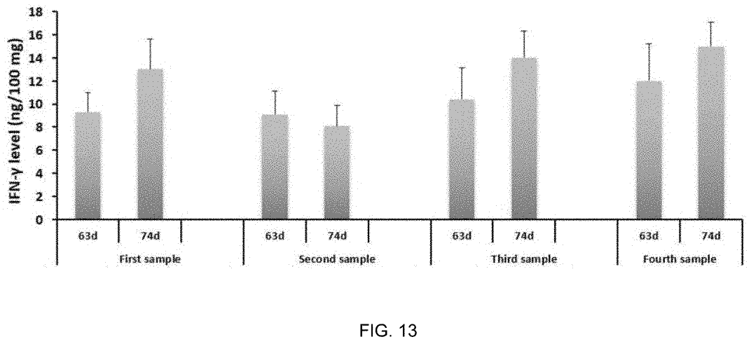

IFN-γ levels were measured systemically and in the lungs and spleen. The results are shown in FIG. 11 to FIG. 13. As shown in FIG. 11, the serum IFN-γ levels increased by administration of MAX composition, but no notable change was seen in other organs, as shown in FIG. 12 and FIG. 13.

The experimental results described above demonstrated that using MAX composition containing four cellulolytic bacteria can inhibit influenza virus.

1. A composition for inhibiting proliferation of influenza virus, comprising:
fermented rice bran powder produced using non-pathogenic microbes that include, as cellulolytic bacteria, at least three Bacillus species, including Bacillus amyloliquefaciens M-4 (deposited under National Institute of Technology and Evaluation (NITE) accession number: NITE P-02538 and NITE BP-02538), Bacillus subtilis M-5 (deposit number: NITE P-02539 and NITE BP-02539) and the Bacillus subtilis-related species Bacillus sp. M-6 (deposit number: NITE P-02940 and NITE P-02940), and at least one Paenibacillus species, including Paenibacillus cineris M-9 (deposit numbers: NITE AP-03304 and NITE BP-03304).
